(19) United States
(12) Patent Application Publication
(10) Pub. No.: US 2026/0306790 A1
(43) Pub. Date: Oct. 8, 2026

(54) Retractable Insertion Tubes for Medical Devices

(71) Applicants: Cassandra M. Piontek, Green Bay, WI (US); Edward W. Piontek, Green Bay, WI (US)

(72) Inventors: Cassandra M. Piontek, Green Bay, WI (US); Edward W. Piontek, Green Bay, WI (US)

(21) Appl. No.: 19/172,357

(22) Filed: Apr. 7, 2025

(51) Int. Cl. A61M 25/00; A61B 10/04; A61B 17/00; A61M 25/01; A61M 25/09

(52) U.S. Cl. CPC A61M 25/002; A61B 10/04; A61M 25/0113; A61M 25/0136; A61M 25/09041; A61B 2017/0034

(57) ABSTRACT

Medical devices including retractable insertion tubes are disclosed herein. An example apparatus for use in a medical procedure includes an annular housing, a drum disposed within the annular housing, a first handle rotatably coupled to the annular housing, the first handle including a notch, a medical device including a second handle, and an insertion tube disposed around the drum, the insertion tube including a first end extending from the notch, a rotation of the drum to cause (1) the insertion tube to uncoil from the drum and (2) the first end to displace from the notch and a second end coupled to the second handle.

### FIELD OF THE DISCLOSURE

This disclosure relates generally to medical devices and, more particularly, to retractable insertion tubes for medical devices.

### BACKGROUND

Some medical devices, like endoscopic biopsy forceps, are used to remove samples from inside body cavities. Endoscopic biopsy forceps generally include a flexible insertion tube, a wire extending through the insertion tube, a removal tool coupled to the wire, and a handle that controls the relative position of the wire and flexible insertion tube. Some such devices can be inserted into the gastrointestinal tract via an endoscope, the removal tool can be positioned around the tissue to be sampled, and the handle manipulated to cause the removal tool to sample tissue. After collecting the sample, the handle is manipulated to secure the sample, and the insertion tube is removed from the body.

### BRIEF DESCRIPTION OF THE DRAWINGS

FIG. 1A is a perspective view of an example first medical device assembly implemented in accordance with the teachings of this disclosure.

FIG. 1B is a side view of the first medical device assembly of FIG. 1A.

FIG. 2 is a perspective view of an interior of the first medical device assembly of FIGS. 1A and 1B.

FIG. 3 is an example second medical device assembly implemented in accordance with the teachings of this disclosure.

FIG. 4 is a flow diagram of example operations that can be used to assemble and use the first medical device of FIGS. 1A-2 and/or the second medical device of FIG. 3.

In general, the same reference numbers will be used throughout the drawing(s) and accompanying written description to refer to the same or like parts. The figures are not necessarily to scale.

### DETAILED DESCRIPTION

An endoscopy is a medical procedure used to examine the inside of a body cavity. During the procedure, an endoscope is inserted into a body cavity and manipulated to view tissues. Endoscopes typically include tools used to examine tissues of interest, such as lights and/or cameras. During some endoscopies, tissue of interest can be sampled (e.g., collected, biopsied, etc.), extracted from the body cavity, and analyzed. Endoscopes for these procedures include channels that enable instruments to be inserted therethrough and sampling or other surgery to be performed. Some such instruments are positioned at the end of a long flexible insertion tube (e.g., greater than a meter, etc.) and controlled via a wire extending therethrough. After collecting the tissue, a medical professional manually retracts the operating instruments from the patient. However, given the length of the flexible insertion tube, the flexible insertion tube can be difficult to control as the flexible insertion tube is being removed from the endoscope. Additionally, excess force applied to the flexible tube during the removal thereof from the endoscope can cause biowaste to be expelled from the endoscope, which can be hazardous to operators of the endoscope.

Examples disclosed herein overcome the above-noted deficiencies and include endoscope-compatible medical devices including retractable insertion tubes. Examples disclosed herein include medical device assemblies that are coupled to a rotatable housing. Example medical device assemblies disclosed herein include insertion tubes coiled about a spool or drum of the medical devices. Some such medical device assemblies disclosed herein can be rotated to control the extension and retraction of an insertion tube through an endoscopic port. Some example medical device assemblies disclosed herein include a motor, which controls the extension and retraction of the insertion tube. Some example medical device assemblies disclosed herein include a heating element, which enables the removal tool attached to the insertion tube of the medical device to be heated to assist with sample collection. Example medical device assemblies disclosed herein enable the precise control of the removal tool during endoscopic procedures and reduce the likelihood of biowaste exposure by medical personnel. Example medical device assemblies disclosed herein are easier and safer to use than prior endoscopic biopsy medical devices.

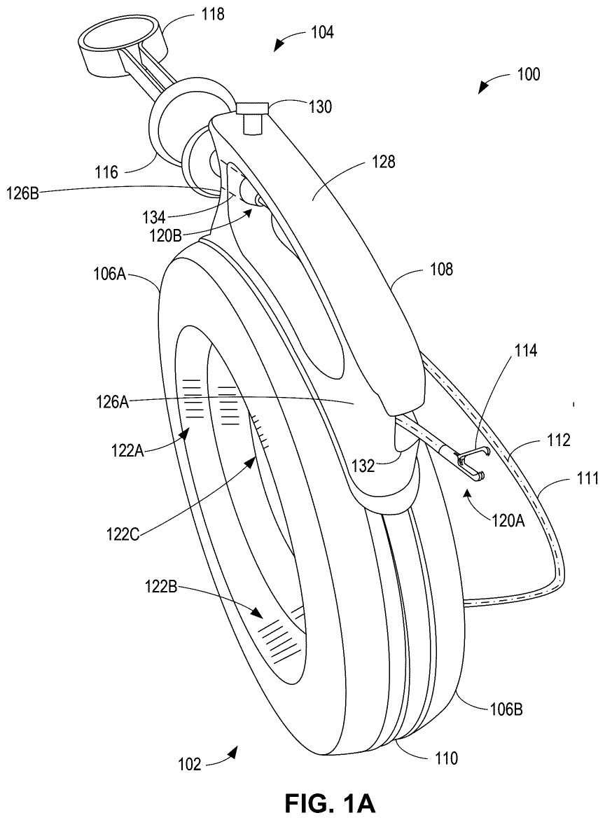

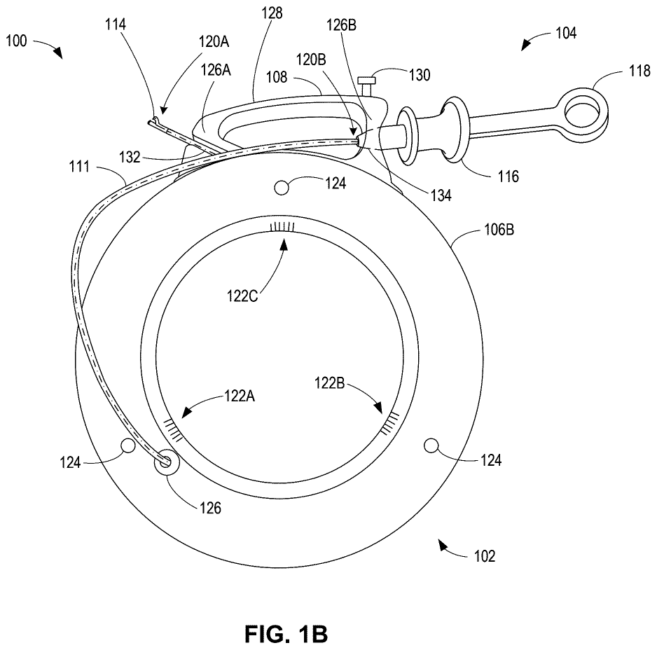

FIG. 1A is a perspective view of an example medical device assembly 100 implemented in accordance with the teachings of this disclosure. FIG. 1B is a side view of the medical device assembly 100 of FIG. 1A. In the illustrated example of FIGS. 1A and 1B, the medical device assembly 100 includes an example housing 102 and an example medical device 104. In the illustrated example of FIG. 1A, the housing 102 includes an example first housing portion 106A, an example second housing portion 106B, an example first handle 108, and an example ring 110. In the illustrated example of FIGS. 1A and 1B, the medical device 104 includes an example insertion tube 111, an example wire 112, an example removal tool 114, an example second handle 116, and an example plunger 118. In the illustrated example of FIGS. 1A and 1B, the insertion tube 111 includes a first end 120A and a second end 120B.

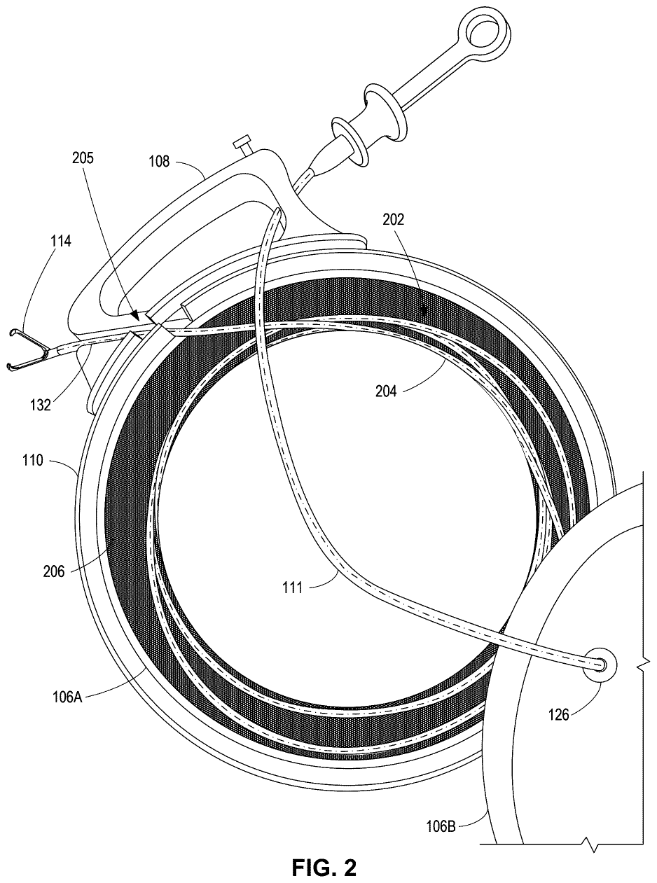

The housing 102 retains the insertion tube 111 during the operation of the medical device assembly 100. The first handle 108 extends radially outward from the outer diameter of the ring 110. In the illustrated example of FIG. 1A, the housing portions 106A, 106B are annular half-shells that, when joined, define an enclosed interior. The interior of the housing portions 106A, 106B is disclosed below in conjunction with FIG. 2. In the illustrated example of FIG. 1B, the housing 102 includes a first grip 122A, a second grip 122B, and a third grip 122C. In the illustrated example of FIG. 1A, the grips 122A, 122B, 122C are grooves (e.g., ridges, etc.) formed in the inner diameter of the housing portions 106A, 106B. The grips 122A, 122B, 122C enable a user to rotate the housing portions 106A, 106B relative to the first handle 108. While the housing 102 includes three grips (e.g., the grips 122A, 122B, 122C, etc.), in other examples, the housing 102 can include a different quantity of grips. In some examples, the gripping function is provided by a materials (e.g., silicone). In some examples, the grips 122A, 122B, 122C and the first handle 108 facilitate use and operation of the first medical device assembly 100. For example, the medical device assembly 100 offers more precise control and more ergometric handling and requires less exertion by a user than conventional endoscopic biopsy devices.

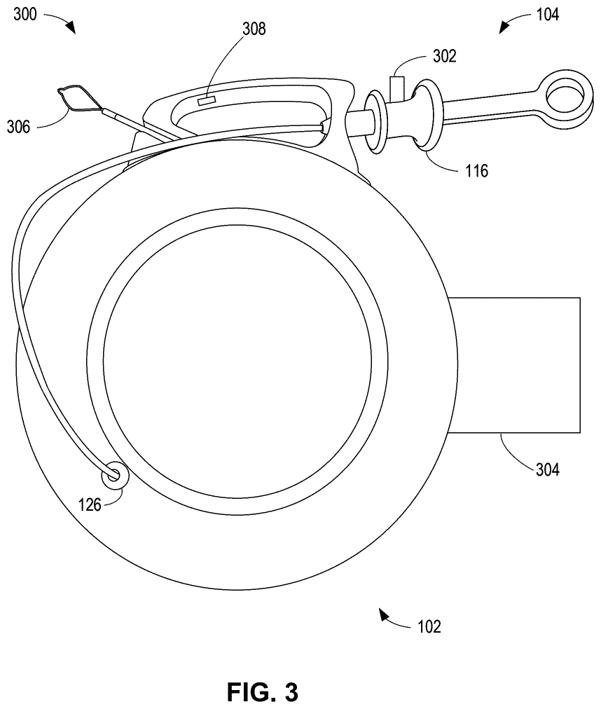

The first handle 108 facilitates the handling of the medical device assembly 100 during the operation thereof. The handle is disclosed below in additional detail in conjunction with FIG. 3. The ring 110 is an annular member positioned between the first housing portion 106A and the second housing portion 106B. In the illustrated example of FIG. 1A, the ring 110 is fixed to the first handle 108. In some examples, the ring 110 and the first handle 108 are an integral component (e.g., monolithic, unitary, molded, etc.). In other examples, the ring 110 and the first handle 108 are rigidly coupled (e.g., via one or more fasteners, via one or more interference fits, via one or more chemical adhesives, etc.). In some examples, the first handle 108 and the ring 110 include (e.g., contain, are composed of, etc.) a medical grade plastic (e.g., polyethylene (PE), polystyrene, (PS), polypropylene (PP), polyvinyl chloride (PVC), polycarbonate (PC), polyetheretherketone (PEEK), polymethylpentene (PMP), acrylonitrile butadiene styrene (ABS), etc.), a metal (e.g., aluminum, stainless steel, titanium, etc.), and/or any other suitable type of material (e.g., silicone, a natural fiber, etc.).

In the illustrated example of FIG. 1A, the ring 110 is disposed between the housing portions 106A, 106B. The housing portions 106A, 106B are releasably coupled via the plurality of fasteners 124. In the illustrated example of FIG. 1B, the ring 110 is retained between the housing portions 106A, 106B via the plurality of fasteners 124 that extend through the housing portions 106A, 106B. In some examples, the fasteners 124 are screws. In other examples, the fasteners 124 can be implemented by any suitable type of fastener. In other examples, the ring 110 can be retained between the housing portions 106A, 106B via one or more interference fit(s), via a chemical adhesive, etc. The coupling of the housing portions 106A, 106B and associated retention of the ring 110 enables the relative rotation of the (1) the ring 110 and the first handle 108 and (2) the housing portions 106A, 106B. In some examples, the relative rotation of the ring 110 and the housing portions 106A, 106B controls the displacement of the removal tool 114 from the first handle 108. The interior of the housing portions 106A, 106B is disclosed below in additional detail in conjunction with FIG. 2.

In the illustrated example of FIG. 1B, the second housing portion 106B includes an example port 126. The port 126 is a through hole that extends between an outer surface of the second housing portion 106B and the interior of the housing 102. In the illustrated example of FIG. 1B, the port 126 is disposed on a side of the second housing portion 106B. In other examples, the port 126 can be disposed at another location of the housing 102 (e.g., on the first housing portion 106A, the outer diameter of the second housing portion 106B, the inner diameter of the second housing portion 106B, etc.).

The medical device 104 is configured to remove a tissue sample (e.g., a polyp, a growth, etc.) from a body cavity. For example, the medical device 104 can be used during a colonoscopy, an esophagogastroduodenoscopy, a cystoscopy, a ureteroscopy, a bronchoscopy, etc. In the illustrated example of FIGS. 1A and 1B, the medical device 104 is an endoscopic biopsy forceps device. It should be appreciated that the teachings of this disclosure are not limited to biopsy forceps. For example, the medical device 104 can be implemented by an endoscopic biopsy forceps snare and/or a medical stone basket device. The coupling between the housing 102 and the medical device 104 is disclosed below in additional detail in conjunction with FIG. 3.

The insertion tube 111 is a flexible tube that extends between the first end 120A and the second end 120B. Throughout this disclosure the term “insertion tube” is used to refer to any medical tubing configured for insertion into a body cavity, such as a catheter, a cannula, a tracheotomy tube, a feeding tube, a drainage tube, a T-tube, etc. As used herein, the insertion tube 111 is also referred to herein to as the “sleeve” or the “outer sleeve.” During an endoscopic procedure, the first end 120A of the insertion tube 111 can be inserted into a port of an endoscope and extended into the body cavity of a patient. In some examples, the outer diameter of the insertion tube 111 includes markings (e.g., measurement markings, etc.), which enables an operator of the medical device assembly 100 to discern the depth of insertion of the insertion tube 111 into an endoscope and/or a body cavity of a patient. In the illustrated example of FIG. 1, the insertion tube 111 enters the interior of the housing 102 via the port 126. In the illustrated example of FIG. 1, the second end 120B of the insertion tube 111 is fixedly coupled to the second handle 116. In other examples, the second end 120B is removably coupled to the second handle 116 (e.g., the second end 120B is removably coupled to the handle 116 via an interference fit, the second end 120B is removably coupled to the handle 116 via a threaded connection, etc.). In some examples, the insertion tube 111 includes (e.g., contains, is composed of, etc.) a flexible medical grade plastic material (e.g., polyethylene, etc.). Additionally or alternatively, the insertion tube 111 includes (e.g., contains, is composed of, etc.) another suitable flexible material (e.g., silicone, etc.). The insertion tube 111 is disposed in the interior of the housing 102. The coupling of the insertion tube 111 is disclosed below in additional detail in conjunction with FIG. 2.

In the illustrated example of FIG. 1A, the wire 112 extends through the interior of the insertion tube 111. In some examples, the wire 112 translates within the interior of the insertion tube 111. That is, the wire 112 can be moved with the insertion tube 111. In the illustrated example of FIG. 1A, the wire 112 is fixedly coupled (e.g., rigidly coupled, etc.) to the removal tool 114 and the plunger 118. In some examples, the wire 112 enables the articulation of the removal tool 114 via the plunger 118. For example, the plunger 118 can be depressed and/or retracted to open and/or close the removal tool 114. In some examples, the wire 112 includes (e.g., contains, is composed of, etc.) one or more filaments of a flexible medical grade metal, such as stainless steel, titanium, and/or a nickel alloy.

In the illustrated example of FIG. 1A, the removal tool 114 is fixedly coupled to the wire 112. The removal tool 114 is configured to extract (e.g., sample, biopsy, etc.) tissue from within a body cavity. In the illustrated example of FIG. 1A, the removal tool 114 is forceps, which can close around and cut a sample. In other examples, the removal tool 114 is a biopsy snare, which can be disposed around a tissue and retracted to extract a sample therefrom. In some examples, the removal tool 114 is continuous with the wire 112 (e.g., the snare and the wire 112 form a continuous flexible member, etc.). In other examples, the removal tool 114 is a stone basket, which can be used to extract a kidney stone and/or other deposits (e.g., other aggregated crystals, mineral build-ups, plaque, etc.). In other examples, the removal tool 114 is a radial jaw and/or a clip. In some examples, the removal tool 114 is absent. In some such examples, the medical device 104 can be inserted for exploratory purposes, to drain a fluid from the body cavity, to administer a fluid, etc. In some such examples, the first end 120A can include a different device, such as a camera, a needle, and/or a light. In some examples, the removal tube 114 is removably coupled to the first end 120A, which enables the removal tube 114 to be interchanged with a different tool after the assembly of the first medical device assembly 100 (e.g., the removal tube 114 is interchangeable, etc.).

The second handle 116 is configured to control the position of the removal tool 114. In the illustrated example of FIG. 1, the plunger 118 is fixedly coupled to the wire 112 and movably coupled to the second handle 116. That is, the plunger 118 can be moved toward the handles 108, 116 (e.g., depressed, etc.) and/or moved away from the handles 108, 116 (e.g., retracted, etc.) to control the position and/or the articulation of the removal tool 114. For example, the actuation of the plunger 118 can cause the opening/closing of the removal tool 114 (e.g., the opening/closing of forceps, etc.) and/or the retraction/extension of the removal tool 114 from the insertion tube 111 (e.g., the retraction/extension of a snare from within the insertion tube 111, etc.). In some examples, the second handle 116 and the plunger 118 include a medical grade plastic, a medical grade metal, silicone, glass, etc. In the illustrated example of FIGS. 1A and 1B, the plunger 118 is a one-finger plunger. In other examples, the plunger 118 is implemented by a two-finger plunger and/or a three-plunger. In some examples, the plunger 118 can include a lock that locks the relative position of the plunger 118 and the second handle 116. In some examples, the lock of the plunger 118 can be engaged to maintain the removal tool 114 during the removal of the insertion tube 111 from an endoscope port.

In the illustrated example of FIGS. 1A and 1B, the first handle 108 includes an example first support 126A, an example second portion 126B, an example grip 128, and an example fastener 130. In the illustrated example of FIGS. 1A and 1B, the first handle 108 includes an example notch 132 disposed in the first support 126A and an example hole 134 disposed in the second support 126B. In the illustrated example of FIG. 1A, the first support 126A and the second support 126B extend from an outer diameter of the ring 110 of the housing 102. In the illustrated example of FIG. 1A, the grip 128 extends between the outboard portion of the supports 126A, 126B.

The notch 132 is a partial opening that extends through the first support 126A. In the illustrated example of FIGS. 1A and 1B, the insertion tube 111 is disposed in and supported by the notch 132 In the illustrated example of FIGS. 1A and 1B, the hole 134 is disposed in the second support 126B. That is, the hole 134 is opposite the notch 132 on the first handle 108. In the illustrated examples of FIGS. 1A and 1B, the second handle 116 is inserted into the hole 134. In the illustrated example of FIGS. 1A and 1B, the fastener 130 extends through the grip 128 and abuts the second handle 116. That is, the fastener 130 retains the second handle 116 of the medical device 104 to the first handle 108 of the housing 102. In the illustrated example of FIGS. 1A and 1B, the fastener 130 is a screw. In other examples, the fastener 130 can be implemented by any suitable type of fastener (e.g., a bolt, a rivet, a leaf spring, etc.). In some examples, the fastener 130 is absent. In some such examples, the medical device 104 is retained in the hole 134 via an interference fit and/or a chemical adhesive. In other examples, the medical device 104 can be maintained in place by a user of the medical device assembly 100.

FIG. 2 is a perspective view of an example interior 202 of the housing 102 of the medical device assembly 100. In the illustrated example of FIG. 2, the fasteners 124 of FIG. 1B have been removed and the second housing portion 106B has been decoupled from the first housing portion 106A. In the illustrated example of FIG. 2, the insertion tube 111 of the medical device 104 of FIG. 1 is coiled (e.g., wrapped, disposed about, etc.) an example drum 204. That is, the insertion tube 111 extends from the first end 120A adjacent to the notch 132, into an example opening 205 in the ring 110, is coiled around an example drum 204, and out of the housing at the port 126. That is, the first end 120A of the insertion tube 111 is fed through the opening 205 of the ring 110 and the notch 132. In the illustrated example of FIG. 2, the opening 205 is on the outer diameter of the ring 110 adjacent to the first handle 108. In the illustrated example of FIG. 2, the opening 205 is aligned with the notch 132 (e.g., the notch 132 is aligned with the opening 205, etc.).

In the illustrated example of FIG. 2, the drum 204 is disposed in the interior 202 of the housing 102. The drum 204 is also referred to herein as a “spool.” In the illustrated example of FIG. 2, the drum 204 is formed by the wall associated with the inner diameter of the housing 102. In some such examples, the drum 204 is formed by the abutment of a first internal wall of the first housing portion 106A and a second internal wall of the second housing portion 106B.

The drum 204 is rigidly coupled to the bodies of the housing portions 106A, 106B. The rigid coupling of the drum 204 to the housing portions 106A, 106B causes the relative rotation of the housing portions 106A, 106B and the ring 110 to correspondingly rotate the drum 204, which coils or uncoils the insertion tube 111 from the drum 204. In some examples, the coiling of the insertion tube 111 about the drum 204 is maintained via the tension of the insertion 111 within the housing 102 between the first end 120A (e.g., friction between the insertion tube 111 and the notch 132, etc.) and the second end 120B (e.g., the rigid coupling of the second end 120B and the second handle 116, etc.). Additionally or alternatively, the port 126 can include a flexible bushing that frictionally retains the insertion tube 111.

As the insertion tube 111 is uncoiled or coiled from the drum 204, the first end 120A of the insertion tube 111 is correspondingly pushed or pulled, respectively, from the notch 132, thereby enabling the displacement of the first end 120A from the notch 132 to be controlled by an operator of the medical device assembly 100. Particularly, rotation in a first direction (e.g., counter-clockwise, etc.) causes the insertion tube 111 to uncoil from the drum 204 and the first end 120A to be displaced from the notch 132 and rotation in a second direction (e.g., clockwise, etc.) causes the insertion tube 111 to coil about the drum 204 and the first end 120A to be displaced toward the notch 132. Accordingly, the rotation of the drum 204 can be used to control the depth of insertion of the first end 120A into an endoscope and/or the body cavity of a patient. In some examples, the insertion tube 111 can be coiled and/or uncoiled from the drum 204 by manually rotating the grips 122A, 122B, 122C relative to the handle 108. In other examples, the housing portions 106A, 106B can be rotated relative to the ring 110 via a motor. An example medical device assembly similar to the medical device assembly 100 of FIGS. 1A-2 including a motor is disclosed below in conjunction with FIG. 3.

In the illustrated example of FIG. 2, the interior 202 includes an example liner 206. The liner 206 is a disposable barrier that shields the interior of the housing 102 from contamination during the extraction of the sample by the removal tool 114 and the insertion tube 111 or other retraction of the insertion tube 111. In some examples, the liner 206 is a flexible plastic sheet disposed between the interior surfaces of the housing 102 (e.g., the drum 204, etc.) and the insertion tube 111. In other examples, the liner 206 is a rigid component that can be coupled to the drum 204 and/or another surface of the housing 102 (e.g., via an adhesive, via an interference fit, etc.). In some examples, after the retraction of the insertion tube 111, the liner 206 can be disposed of and replaced, which enables the housing 102 to be reused. In other examples, the liner 206 is absent. In some such examples, the housing 102 can be sanitized before reuse. In other examples, the housing 102 is a single-use disposable item.

FIG. 3 is a second example medical device assembly 300 implemented in accordance with the teachings of this disclosure. The second medical device assembly 300 is similar to the first medical device assembly 100 of FIGS. 1A-2, except the second medical device assembly 300 includes an example heat element 302, an example motor 304, and an example removal tool 306 and as otherwise noted. In the illustrated example of FIG. 3, the second medical device assembly 300 includes the housing 102 of FIG. 1 and the medical device 104 of FIG. 1. In the illustrated example of FIG. 3, the removal tool 306 is a biopsy snare. In other examples, the removal tool 306 can be implemented by the removal tool 114 of FIGS. 1A and 1B and/or a different type of biopsy tool.

The heat element 302 is a mechanism that increases the temperature of the removal tool 306. For example, the heat element 302 can be an electrical source (e.g., a battery, an outlet coupled to a power source of the motor 304, etc.) that generates a current through the wire 112 and the removal tool 306 (e.g., the removal tool 306 is heated via electrocautery, etc.). In such examples, the removal tool 306 is heated via the current flowing therethrough. In other examples, the heat element 302 can be implemented by any other suitable mechanism, such as a resistance wire heating element that transfers heat to the wire 112 via conduction, a chemical heater, etc. In the illustrated example of FIG. 3, the heat element 302 is disposed in the second handle 116. In other examples, the heat element 302 is disposed at a different location (e.g., in the housing 102, etc.). In some examples, the heat element 302 is absent and the removal tool 306 is heated prior to being inserted into the endoscope.

The motor 304 provides automatic extension and/or retraction of the insertion tube 111 without manual articulation of the grips 122A, 122B, 122C by a user of the medical device assembly 300. The motor 304 provides torque to rotate the housing 102 relative to the ring 110. That is, the motor 304 provides power to change the relative position of the first end 120A of the insertion tube 111 and automatically extends (e.g., deploys) and/or retracts the insertion tube 111 from an endoscope and/or body cavity of a patient. In the illustrated example of FIG. 3, the motor 304 is an electric motor. In some examples, the motor 304 is powered by a battery of the medical device assembly 100. In other examples, the motor 304 can be powered via a connection to an external power source (e.g., a wall outlet, etc.). In other examples, the motor 304 can be implemented by a pneumatic and/or a hydraulic motor. In some examples, the motor 304 is electrically coupled to a switch 308, which controls the rotation of the motor 304. In the illustrated example of FIG. 3, the switch 308 is disposed on the first handle 108. In some examples, the motor 304 is absent. In such examples, the housing 102 of the medical device 104 can be rotated manually by a user of the first medical device assembly 100.

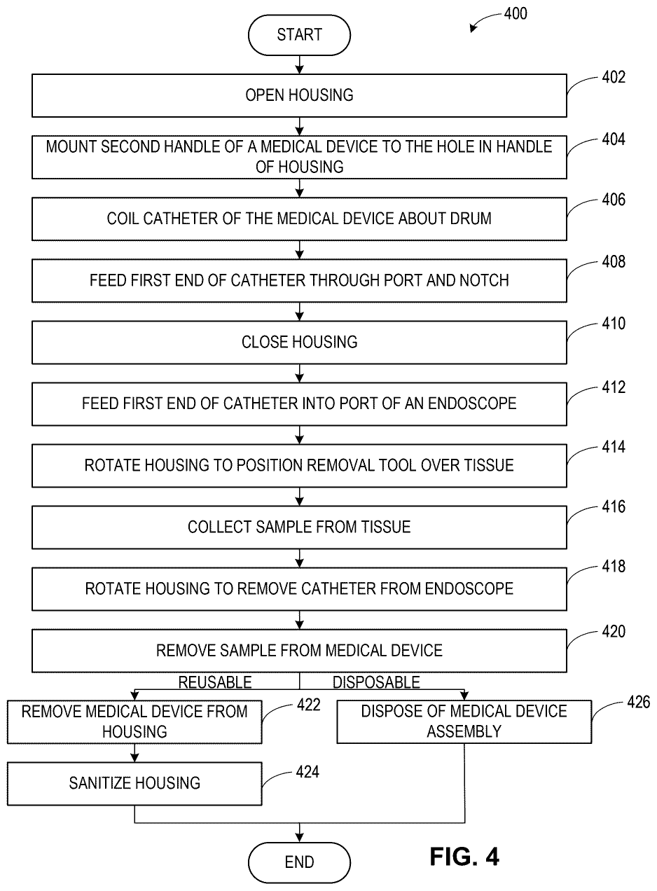

FIG. 4 is a flow diagram of example operations 400 that can be used to assemble and operate the first medical device assembly 100 of FIGS. 1A-2 and/or the second medical device assembly 300 of FIG. 3. Blocks 402-410 relate to the assembly of the first medical device assembly 100 of FIGS. 1A-2 and/or the second medical device assembly 300 of FIG. 3. Blocks 412-424 relate to the use of the first medical device assembly 100 of FIGS. 1A-2 and/or the second medical device assembly 300 of FIG. 3 during an endoscopy. It should be appreciated the medical device assemblies 100, 300 can be used in other medical procedures, such as an esophagogastroduodenoscopy, a cystoscopy, and/or a ureteroscopy. It should also be appreciated that assembly of the first medical device assembly 100 and/or the second medical device assembly 300 (blocks 402-410), use of the first medical device assembly 100 and/or the second medical device assembly 300 (blocks 412-422), and sanitation of the first medical device assembly 100 and/or the second medical device assembly (block 424) can each and/or all be performed by one or more different entities or operators at one or more different locations.

The operations 400 begin at block 402. At block 402, the housing 102 is opened. For example, the fasteners 124 of FIG. 1B can be removed from the housing portions 106A, 106B and the housing portions 106A, 106B can be separated. In other examples, the housing portions 106A, 106B can be separated in any other suitable manner (e.g., applying a force to overcome an interference fit retaining the housing portions 106A, 106B, rotating the housing portions 106A, 106B to unthread them, etc.). At block 404, the second handle 116 of the medical device 104 is mounted in the hole 134. For example, the second handle 116 can be inserted into the hole 134 and retained therein by tightening the fastener 130. In other examples, the second handle 116 can be mounted in the hole 134 via an interference fit and/or a chemical adhesive. At block 406, the insertion tube 111 of the medical device 104 is coiled about the drum 204. For example, the insertion tube 111 can be manually wrapped around the drum 204 and positioned on the liner 206. At block 408, the first end 120A of the insertion tube 111 of the medical device 104 is fed through the port 126 and the notch 132. For example, the first end 120A of the insertion tube 111 can be fed through the port 126 and the notch 132 such that the removal tool 114 extends slightly from the first handle 108. At block 410, the housing 102 is closed. For example, the fasteners 124 can be installed to join the housing portions 106A, 106B.

At block 412, the first end 120A of the insertion tube 111 is fed into an endoscope. For example, the first end 120A can be manually inserted into a port of an endoscope. At block 414, the housing 102 is rotated until the removal tool 114 is positioned over a tissue from which a sample is to be collected. For example, the housing 102 can be manually rotated while holding the first handle 108. In other examples, the housing 102 can be rotated by operating an electric motor (e.g., the motor 304 of FIG. 3, etc.). In some examples, the rotation of the housing 102 uncoils the insertion tube 111 from around the drum 204 and displaces the first end 120A into the endoscope. At block 416, a sample is collected from the tissue. For example, a user of the medical device assembly 100 can collect a sample by articulating the removal tool 114 via the plunger 118.

At block 418, the housing 102 is rotated until the insertion tube 111 is removed from the endoscope. For example, the housing 102 can be manually rotated (e.g., in an opposite direction as block 414, etc.) while holding the first handle 108. In other examples, the housing 102 can be rotated by operating an electric motor (e.g., the motor 304 of FIG. 3, etc.). In some examples, the rotation of the housing 102 coils the insertion tube 111 from around the drum 204 and displaces the first end 120A toward the medical device assembly 100. The rotation of the housing 102 and coiling of the insertion tube 111 about the drum 204 enables a user to precisely control the insertion tube 111 during removal from a body cavity, which reduces the likelihood of an operator applying excess force to the insertion tube 111 during the removal. In some examples, the reduced likelihood of applying excess force to the insertion tube 111 decreases the likelihood of unintentional exposure of medical personnel to biowaste as the first end 120A is removed from the endoscope port. The reduction in the likelihood of exposure to biowaste increases the safety of operation of the medical device assemblies 100, 300 when compared to conventional endoscopic biopsy devices.

At block 420, the sample is removed from the removal tool 114. For example, the plunger 118 can be articulated to release the sample into a secure vessel (e.g., a test tube, a sample vial, etc.). If the housing 102 is reusable, the operations 400 advance to block 424. If the housing 102 is disposable (e.g., the housing 102 is not reused between procedures, etc.), the operations 400 advance to block 426. At block 422, the medical device 104 is removed from the housing 102. For example, the housing 102 can be opened, the insertion tube 111 can be uncoiled from the drum 204, the insertion tube 111 can be removed from the notch 132 and the port 126, and the second handle 116 can be removed from the hole 134. At block 424, the housing 102 is sanitized. For example, the liner 206 can be removed and replaced. Additionally or alternatively, the body of the housing 102 can be cleaned, sterilized in an autoclave, sterilized with induction heating, etc. At block 426, the housing 102 is discarded (e.g., the housing 102 is placed in a biohazard container for later disposal, etc.).

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities, etc., the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities, etc., the phrase “at least one of A or B” is intended to refer to implementations including any of (1) at least one A, (2) at least one B, or (3) at least one A and at least one B.

As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements, or actions may be implemented by, e.g., the same entity or object. Additionally, although individual features may be included in different examples or claims, these may possibly be combined, and the inclusion in different examples or claims does not imply that a combination of features is not feasible and/or advantageous.

As used herein, connection references (e.g., attached, coupled, connected, and joined) may include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly within the context of the discussion (e.g., within a claim) in which the elements might, for example, otherwise share a same name.

As used herein, “approximately” and “about” modify their subjects/values to recognize the potential presence of variations that occur in real world applications. For example, “approximately” and “about” may modify dimensions that may not be exact due to manufacturing tolerances and/or other real world imperfections as will be understood by persons of ordinary skill in the art. For example, “approximately” and “about” may indicate such dimensions may be within a tolerance range of +/−10% unless otherwise specified herein.

Retractable insertion tubes for medical devices are disclosed herein. Further examples and combinations thereof include the following:

Example 1 includes an apparatus for use in a medical procedure, the apparatus comprising an annular housing, a drum disposed within the annular housing, a first handle rotatably coupled to the annular housing, the first handle including a notch, a medical device including a second handle, and an insertion tube disposed around the drum, the insertion tube including a first end extending from the notch, a rotation of the drum to cause (1) the insertion tube to uncoil from the drum and (2) the first end to displace from the notch, and a second end coupled to the second handle.

Example 2 includes the apparatus of any example 1, wherein the first end includes forceps.

Example 3 includes the apparatus of any one or more of examples 1-2, wherein the first end includes a snare.

Example 4 includes the apparatus of any one or more of examples 1-3, wherein the first end is a stone basket.

Example 5 includes the apparatus of any one or more of examples 1-4, further including a motor to rotate the annular housing relative to the first handle.

Example 6 includes the apparatus of any one or more of examples 1-5, wherein the medical device includes a wire disposed the insertion tube, and a plunger coupled to the wire, the plunger to control a position of the wire relative to the insertion tube.

Example 7 includes the apparatus of any one or more of examples 1-6, wherein the rotation of the drum to uncoil the insertion tube is in a first direction and wherein rotation of the drum in a second direction is to cause (1) the insertion tube to coil around the drum and (2) the first end to move toward the notch.

Example 8 includes the apparatus of any one or more of examples 1-7, wherein the second handle is removably coupled to the first handle.

Example 9 includes the apparatus of any one or more of examples 1-8, wherein the first handle includes a hole, the second handle disposed within the hole.

Example 10 includes the apparatus of any one or more of examples 1-9, wherein the hole is opposite the notch.

Example 11 includes the apparatus of any one or more of examples 1-10, wherein the second end is rigidly coupled to the second handle within the hole.

Example 12 includes the apparatus of any one or more of examples 1-11, wherein the insertion tube includes a body between the first end and the second end and further including a liner disposed between the body and the annular housing.

Example 13 includes a medical apparatus comprising a first housing portion, a second housing portion coupled to the first housing portion, the second housing portion including a drum, a ring between the first housing portion and the second housing portion, the ring rotatable relative to the first housing portion, an insertion tube including a removal tool, the insertion tube coiled about the drum, the insertion tube extending from an opening in the ring, a relative rotation of the first housing portion and the ring to cause the insertion tube to uncoil from the drum and to extend from the ring.

Example 14 includes the medical apparatus of example 13, wherein the ring includes a first support extending radially outboard from the first housing portion, a second support extending radially outboard from the second housing portion, and a grip extending between the first support and the second support.

Example 15 includes the medical apparatus of any one or more of examples 13-14, wherein the first support includes a notch aligned with the opening, the insertion tube disposed in the notch.

Example 16 includes the medical apparatus of any one or more of examples 13-15, wherein the second support includes a hole, the removal tool is disposed at a first end of the insertion tube, and the apparatus further includes a handle disposed in the hole, the handle coupled to a second end of the insertion tube.

Example 17 includes the medical apparatus of any one or more of examples 13-16, further including a plunger movably coupled to the handle, and a wire within the insertion tube, the wire coupling the plunger to the removal tool.

Example 18 includes the medical apparatus of any one or more of examples 13-17, wherein the insertion tube extends through a port on the second housing portion.

Example 19 includes a method comprising inserting an end of an insertion tube into an endoscope, the insertion tube extending from a notch of a handle radially outboard of the housing, the insertion tube coiled about a drum of in an interior of the housing, rotating the housing relative to the handle, the rotation of the housing to uncoil the insertion tube from the drum and extend the insertion tube into the endoscope.

Example 20 includes the method of example 19, further including sampling a tissue via a plunger coupled to the first handle, and rotating the housing to retract the insertion tube from the endoscope.

The following claims are hereby incorporated into this Detailed Description by this reference. Although certain example systems, apparatus, articles of manufacture, and methods have been disclosed herein, the scope of coverage of this patent is not limited thereto. On the contrary, this patent covers all systems, apparatus, articles of manufacture, and methods fairly falling within the scope of the claims of this patent.

What is claimed is:

1. An apparatus for use in a medical procedure, the apparatus comprising:
an annular housing;
a drum disposed within the annular housing;
a first handle rotatably coupled to the annular housing, the first handle including a notch;
a medical device including:
a second handle; and
an insertion tube disposed around the drum, the insertion tube including:
a first end extending from the notch, a rotation of the drum to cause (1) the insertion tube to uncoil from the drum and (2) the first end to displace from the notch; and
a second end coupled to the second handle.

2. The apparatus of claim 1, wherein the first end includes forceps.

3. The apparatus of claim 1, wherein the first end includes a snare.

4. The apparatus of claim 1, wherein the first end is a stone basket.

5. The apparatus of claim 1, further including a motor to rotate the annular housing relative to the first handle.

6. The apparatus of claim 1, wherein the medical device includes:
a wire disposed the insertion tube; and
a plunger coupled to the wire, the plunger to control a position of the wire relative to the insertion tube.

7. The apparatus of claim 1, wherein the rotation of the drum to uncoil the insertion tube is in a first direction and wherein a rotation of the drum in a second direction is to cause (1) the insertion tube to coil around the drum and (2) the first end to move toward the notch.

8. The apparatus of claim 1, wherein the second handle is removably coupled to the first handle.

9. The apparatus of claim 8, wherein the first handle includes a hole, the second handle disposed within the hole.

10. The apparatus of claim 9, wherein the hole is opposite the notch.

11. The apparatus of claim 9, wherein the second end is rigidly coupled to the second handle within the hole.

12. The apparatus of claim 1, wherein the insertion tube includes a body between the first end and the second end and further including a liner disposed between the body and the annular housing.

13. A medical apparatus comprising:
a first housing portion;
a second housing portion coupled to the first housing portion, the second housing portion including a drum;
a ring between the first housing portion and the second housing portion, the ring rotatable relative to the first housing portion;
an insertion tube including a removal tool, the insertion tube coiled about the drum, the insertion tube extending from an opening in the ring, a relative rotation of the first housing portion and the ring to cause the insertion tube to uncoil from the drum and to extend from the ring.

14. The medical apparatus of claim 13, wherein the ring includes:
a first support extending radially outboard from the first housing portion;
a second support extending radially outboard from the second housing portion; and
a grip extending between the first support and the second support.

15. The medical apparatus of claim 14, wherein the first support includes a notch aligned with the opening, the insertion tube disposed in the notch.

16. The medical apparatus of claim 14, wherein the second support includes a hole, the removal tool is disposed at a first end of the insertion tube, and the medical apparatus further includes a handle disposed in the hole, the handle coupled to a second end of the insertion tube.

17. The medical apparatus of claim 16, further including:
a plunger movably coupled to the handle; and
a wire within the insertion tube, the wire coupling the plunger to the removal tool.

18. The medical apparatus of claim 13, wherein the insertion tube extends through a port on the second housing portion.

19. A method comprising:
inserting an end of an insertion tube into an endoscope, the insertion tube extending from a notch of a handle radially outboard of a housing, the insertion tube coiled about a drum of in an interior of the housing;
rotating the housing relative to the handle, the rotation of the housing to uncoil the insertion tube from the drum and extend the insertion tube into the endoscope.

20. The method of claim 19, further including:
sampling a tissue via a plunger coupled to the housing; and
rotating the housing to retract the insertion tube from the endoscope.
